(19) United States
(12) Patent Application Publication
(10) Pub. No.: US 2026/0144670 A1
(43) Pub. Date: May 28, 2026

(54) Cold and Hot Compress Device

(71) Applicant: Xiamen Ubest Electronic Technology Co., Ltd., Xiamen (CN)

(72) Inventor: Zonghu Zhu, Xiamen (CN)

(21) Appl. No.: 19/015,615

(22) Filed: Jan. 9, 2025

(30) Foreign Application Priority Data
Nov. 27, 2024 (CN) 202422904505.2
Dec. 26, 2024 (CN) 202423234976.3

(51) Int. Cl. A61F 7/00

(52) U.S. Cl. CPC A61F 7/0085; A61F 2007/0018; A61F 2007/0023; A61F 2007/003; A61F 2007/0031; A61F 2007/0032; A61F 2007/0034; A61F 2007/0035; A61F 2007/0036; A61F 2007/0039; A61F 2007/0054

(57) ABSTRACT

A cold and hot compress device includes a carrier and a main machine, wherein a cooling and heating assembly and a circulation assembly are provided in the main machine, and the cooling and heating assembly includes a compressor, a heat exchanger and a throttling element, wherein the compressor, the heat exchanger and the throttling element are in communication; and the circulation assembly includes a storage tank, a pumping device and a delivery pipe, wherein the pumping device is connected to the storage tank and the carrier via the delivery pipe, and the delivery pipe exchanges heat with the cooling and heating assembly; and compared with traditional semiconductor cooling and heating, the cooling and heating assembly composed of a compressor, a heat exchanger and a throttling element has wider application scenes and a larger load, which can be applied to large-area and high-power cold and hot compress operation.

### CROSS-REFERENCE TO RELATED APPLICATIONS

This application claims priority to Chinese Patent Application No. 202422904505.2, filed on Nov. 27, 2024 and Chinese Patent Application No. 202423234976.3, filed on Dec. 26, 2024, the content of which is incorporated herein by reference in its entirety.

### TECHNICAL FIELD

The present application relates to the field of care equipment, and more particularly to a cold and hot compress device.

### BACKGROUND

The cold and hot compress device generally transfers a temperature of a carrier through an assembly capable of cooling and heating, such that a care site on which the carrier is attached can be cold and hot compressed to achieve an effect of therapeutic care. In the design of an existing cold and hot compress device, semiconductor cooling and heating is commonly used, but the semiconductor is mostly used in small cold and hot compress device. In the case of large area cold and hot compress, it cannot meet rapid cooling and heating; even if multiple groups of semiconductors are combined to form a working surface with a large breadth, there are limitations to a semiconductor structure that cannot be bent arbitrarily and cannot meet a close fit to the care site.

### SUMMARY

The technical problem to be solved by the present application is providing a cold and hot compress device that can be applied to large-area cooling and heating.

In order to solve the above-mentioned technical problem, the technical solution adopted by the present application is a cold and hot compress device, wherein a cooling and heating assembly and a circulation assembly are provided in the main machine, and the cooling and heating assembly includes a compressor, a heat exchanger and a throttling element, wherein the compressor, the heat exchanger and the throttling element are in communication; and the circulation assembly includes a storage tank, a pumping device and a delivery pipe, wherein the pumping device is connected to the storage tank and the carrier via the delivery pipe, and the delivery pipe exchanges heat with the cooling and heating assembly.

Further, a heat dissipation assembly is provided within the main machine, the heat dissipation assembly includes a radiator and a heat dissipation fan, one side of the main machine is provided with the radiator, and one side of the radiator is provided with the heat dissipation fan.

Further, the storage tank includes a first tank and a second tank, the first tank is in communication with the second tank, and the second tank is in communication with the pumping device and the carrier.

Further, the cooling and heating assembly further includes a heating element configured for heating a medium in the delivery pipe.

Further, a channel of the delivery pipe includes an internal circulation pipe, a first circulation pipe and a second circulation pipe, the internal circulation pipe is provided in the main machine, and the carrier, the pumping device and the storage tank are all connected via the internal circulation pipe; and the first circulation pipe and the second circulation pipe connect the inner circulation pipe and the carrier.

Further, the delivery pipe further includes a communication pipe; an air pump is provided in the main machine, having one end connected with an air valve, and an expansion layer is provided on the carrier; and the air pump, the air valve and the expansion layer of the carrier are all connected via a communication pipe.

Further, a three-way valve is provided in the main machine; the pumping device is a gas-liquid dual-purpose pump; a first end of the three-way valve is connected with the storage tank; a second end of the three-way valve is connected with atmosphere; and a third end of the three-way valve is connected with the pumping device.

Further, a mounting height of the first tank is higher than a mounting height of the second tank.

Further, the expansion layer includes a plurality of expansion units, each of which is connected with one air valve; and the plurality of air valves are connected with the air pump.

Further, the air valves are provided on the carrier.

Further, the carrier includes a body for integrally wrapping legs, shoulders, a waist, arms, an upper body or a whole body and a connecting structure, and the body is provided with the connecting structure.

The beneficial effects of the present application are as follows: through the arrangement of the circulation assembly, the medium is pumped by the storage tank via the pumping device, enters into the carrier to exchange heat with a care site after exchanging heat with the cooling and heating assembly, and finally returns to the storage tank; a temperature control of the medium in the delivery pipe is realized through cooling and heating by the cooling and heating assembly, so that a user can realize a cold and hot compress function after assembling the carrier; compared with traditional semiconductor cooling and heating, the cooling and heating assembly composed of the compressor, the heat exchanger and the throttling element has wider application scenes and a larger load, which can be applied to large-area and high-power cold and hot compress operation.

### BRIEF DESCRIPTION OF THE DRAWINGS

FIG. 1 is a schematic structural diagram of a cold and hot compress device of a detailed implementation of the present application;

FIG. 2 is a schematic structural diagram of a working layer of a carrier of the cold and hot compress device of a detailed implementation of the present application;

FIG. 3 is a schematic structural diagram of an expansion layer of the carrier of the cold and hot compress device of a detailed implementation of the present application;

FIG. 4 is a schematic structural diagram of a first tank and a second tank of the cold and hot compress device of a detailed implementation of the present application;

FIG. 5 is a schematic structural diagram of another perspective of the first tank and the second tank of the cold and hot compress device of a detailed implementation of the present application;

FIG. 6 is a schematic structural diagram of a housing of a main machine of the cold and hot compress device of a detailed implementation of the present application;

FIG. 7 is a schematic structural diagram of an interior of the main machine of the cold and hot compress device of a detailed implementation of the present application;

FIG. 8 is a schematic structural diagram of connecting a pumping device and a three-way valve of the cold and hot compress device of a detailed implementation of the present application;

FIG. 9 is a sectional view of a pipeline structure integrally formed by combination of a first circulation pipe, a communication pipe and a second circulation pipe of the cold and hot compress device of a detailed implementation of the present application;

FIG. 10 is a schematic structural diagram of a pipeline structure formed by combination of the first circulation pipe, the communication pipe and the second circulation pipe in connecting the main machine with a single carrier of the cold and hot compress device of a detailed implementation of the present application;

FIG. 11 is a schematic structural diagram of a pipeline structure with a shunting function, formed by combination of the first circulation pipe, the communication pipe and the second circulation pipe, in connecting the main machine with a plurality of carriers of the cold and hot compress device of a detailed implementation of the present application;

FIG. 12 is a schematic structural diagram of an expansion unit of an expansion layer of a leg binding carrier of the cold and hot compress device of a detailed implementation of the present application;

FIG. 13 is a schematic structural diagram of an air valve arranged on the leg binding carrier of the cold and hot compress device of a detailed implementation of the present application;

FIG. 14 is a schematic structural diagram of the leg binding carrier in use of the cold and hot compress device of a detailed implementation of the present application;

FIG. 15 is a schematic structural diagram of an arm binding carrier in use of the cold and hot compress device of a detailed implementation of the present application;

FIG. 16 is a schematic structural diagram of a waist binding carrier in use of the cold and hot compress device of a detailed implementation of the present application;

FIG. 17 is a schematic structural diagram of an upper body binding carrier in use of the cold and hot compress device of a detailed implementation of the present application;

FIG. 18 is a schematic structural diagram of a sleeping bag in use of the cold and hot compress device of a detailed implementation of the present application; and

FIG. 19 is a schematic structural diagram of another style of sleeping bag in use of the cold and hot compress device of a detailed implementation of the present application;

### DESCRIPTION OF THE REFERENCE NUMERALS

1, carrier; 11, expansion layer; 111, expansion unit; 12, working layer; 13, body; 14, connecting structure; 2, main machine; 21, compressor; 22, condenser; 23, control circuit board; 24, pumping device; 25, radiator; 26, heat dissipation fan; 27, first circulation pipe; 28, second circulation pipe; 29, communication pipe; 30, air pump; 31, air valve; 32, quick connector; 33, display screen; 34, horn; 35, power source; 36, power source interface; 37, switch; 38, first tank; 39, second tank; 40, three-way valve; 41, internal circulation pipe; 42, heating element; 43, plate type heat exchanger; 44, control line; 45, throttling element.

### DETAILED DESCRIPTION OF THE EMBODIMENTS

In order to explain the technical contents, the achieved objects and the effects of the present application in detail, the following description is made in conjunction with the implementations and the accompanying drawings.

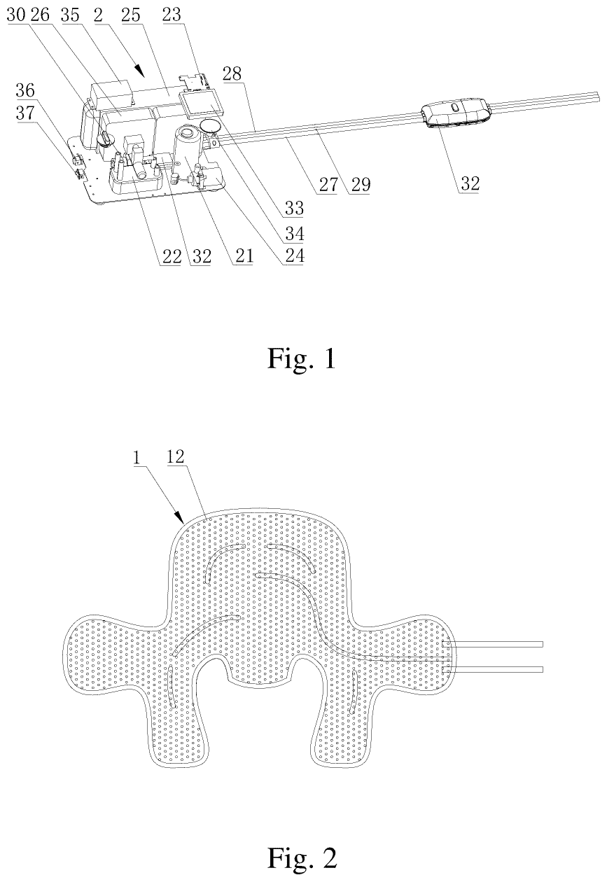

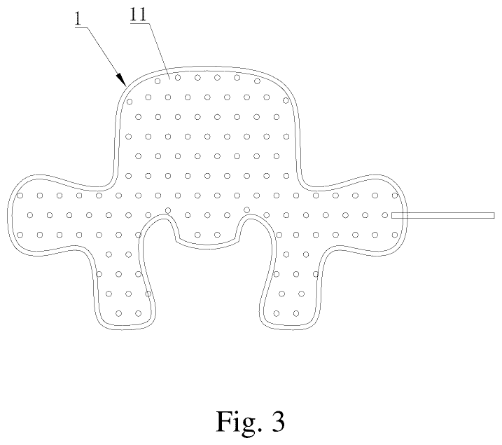

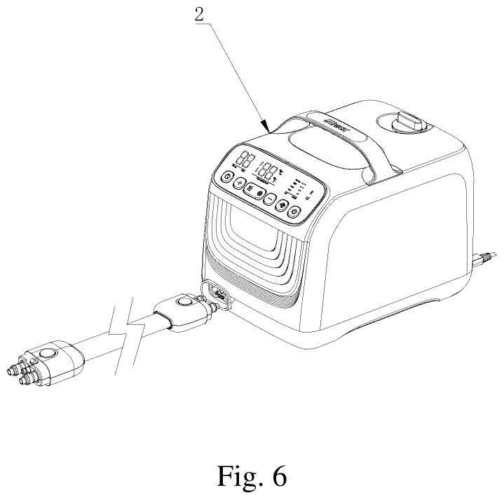

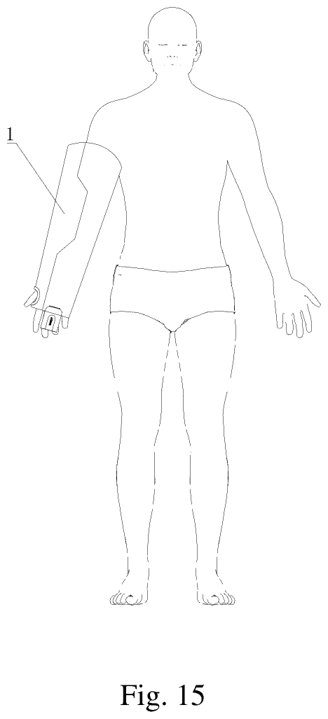

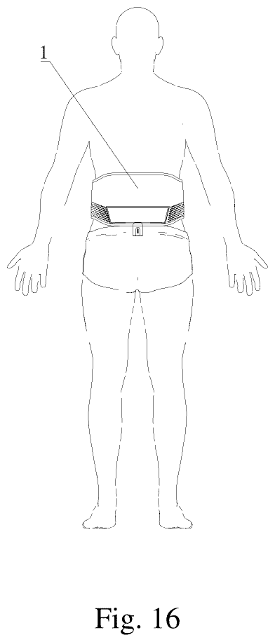

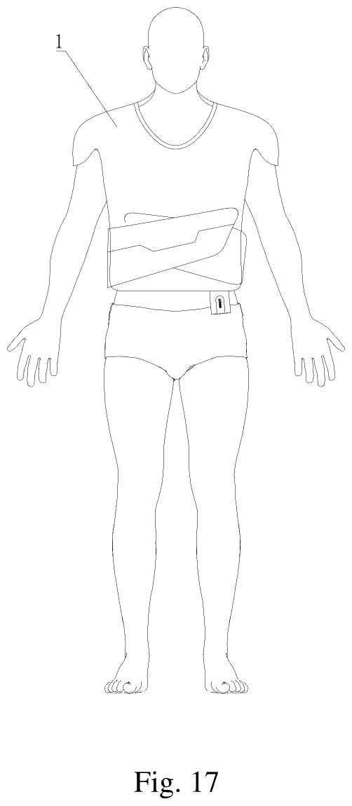

With reference to FIGS. 1 to 19, a cold and hot compress device includes a carrier 1 and a main machine 2, wherein a cooling and heating assembly and a circulation assembly are provided in the main machine 2, and the cooling and heating assembly includes a compressor 21, a heat exchanger and a throttling element 45, wherein the compressor 21, the heat exchanger and the throttling element 45 are in communication; and the circulation assembly includes a storage tank, a pumping device 24 and a delivery pipe, wherein the pumping device 24 is connected to the storage tank and the carrier 1 via the delivery pipe, and the delivery pipe exchanges heat with the cooling and heating assembly.

It can be seen from the foregoing description that the beneficial effects of the present application are as follows: through the arrangement of the circulation assembly, the medium is pumped by the storage tank via the pumping device 24, enters into the carrier 1 to exchange heat with a care site after exchanging heat with the cooling and heating assembly, and finally returns to the storage tank; a temperature control of the medium in the delivery pipe is realized through cooling and heating by the cooling and heating assembly, so that a user can realize a cold and hot compress function after assembling the carrier 1; compared with traditional semiconductor cooling and heating, the cooling and heating assembly composed of the compressor 21, the heat exchanger and the throttling element 45 has wider application scenes and a larger load, which can be applied to large-area and high-power cold and hot compress operation.

Further, a heat dissipation assembly is provided within the main machine 2, the heat dissipation assembly includes a radiator 25 and a heat dissipation fan 26, one side of the main machine 2 is provided with the radiator 25, and one side of the radiator 25 is provided with the heat dissipation fan 26.

As can be seen from the above, by using a combination of the heat dissipation fan 26 and the radiator 25, it is possible to actively and quickly discharge the working heat in the main machine 2, avoiding an influence on a temperature of the medium in the delivery pipe.

Further, the storage tank includes a first tank 38 and a second tank 39, the first tank 38 is in communication with the second tank 39, and the second tank 39 is in communication with the pumping device 24 and the carrier 1.

It can be seen from the foregoing description that when the main machine is in communication with a single carrier 1, it can satisfy circulation supply through a certain amount of medium stored in the second tank 39; when the main machine is in communication with a plurality of carriers 1, the medium in the separate second tank 39 cannot satisfy the circulation supply, and at this time, the medium is replenished into the second tank 39 through the first tank 38 for adjustment and improvement. Meanwhile, when the second tank 39 is used to make a small circulation, a single carrier 1 can be rapidly heated and cooled.

Further, the cooling and heating assembly further includes a heating element 42 configured for heating a medium in the delivery pipe.

It can be seen from the foregoing description that the heating element 42 is used to achieve a heating function of the cooling and heating assembly, and alternatively, an electric heating wire can be used as the heating element. In addition, a combination of the compressor 21, a condenser 22, an expansion valve and an evaporator realize a cooling function of the cooling and heating assembly; and the heating function and the cooling function are respectively controlled and realized by two assemblies.

Further, a channel of the delivery pipe includes an internal circulation pipe 41, a first circulation pipe 27 and a second circulation pipe 28, the internal circulation pipe 41 is provided in the main machine 2, and the carrier 1, the pumping device 24 and the storage tank are all connected via the internal circulation pipe; and the first circulation pipe 27 and the second circulation pipe 28 connect the inner circulation pipe 41 and the carrier 1.

It can be seen from the foregoing description that the carrier 1, the pumping device 24 and the storage tank are connected via the internal circulation pipe 41, and the first circulation pipe 27 and the second circulation pipe 28 serve as connection pipes of the internal circulation pipe 41 and the carrier 1 and are configured for output and input of the medium in the carrier 1.

Further, the delivery pipe further includes a communication pipe 29; an air pump 30 is provided in the main machine 2, having one end connected with an air valve 31, and an expansion layer 11 is provided on the carrier 1; and the air pump 30, the air valve 31 and the expansion layer 11 of the carrier 1 are all connected via the communication pipe 29.

It can be seen from the foregoing description that a filling gas is output through the air pump 30 and the air valve 31, and is poured into the expansion layer 11 of the carrier 1 via the communication pipe 29 so that the carrier 1 is expanded to tightly adhere to the care site.

Further, a three-way valve 40 is provided in the main machine 2, the pumping device 24 is a gas-liquid dual-purpose pump; a first end of the three-way valve 40 is connected with the storage tank; a second end of the three-way valve 40 is connected with atmosphere; and a third end of the three-way valve 40 is connected with the pumping device 24.

It can be seen from the foregoing description that, by the design of the three-way valve 40 in the main machine 2 and application of the gas-liquid dual-purpose pump, when it is necessary to discharge the medium inside the carrier 1, gas can be pumped by the air pump 30 through open and close control of the three-way valve 40 to achieve that.

Further, a mounting height of the first tank 38 is higher than a mounting height of the second tank 39.

It can be seen from the foregoing description that by providing the heights of the first tank 38 and the second tank 39, when the medium in the second tank 39 is insufficient to support the circulation supply, the medium in the first tank 38 can quickly flow into the second tank 39 driven by a self-weight.

Further, the expansion layer 11 includes a plurality of expansion units 111, each of which is connected with one air valve; and the plurality of air valves 31 are connected with the air pump 30.

It can be seen from the foregoing description that by the designs of the plurality of expansion units 111 and air valve 31, a human body part in a region covered by the carrier 1 can be regionally pressed by air pressure step by step, so that blood in the human body part can flow back rapidly, to improve a congestion state of the part after exercise, reduce fatigue after exercise, and accelerate repair of the part.

Further, the air valves 31 are provided on the carrier 1.

It can be seen from the foregoing description that through the arrangement of the air valves 31 on the carrier 1, mounting space occupation on the main machine 2 can be reduced, and a sufficient mounting space can be reserved inside the main machine 2 to configure other components.

Further, the carrier 1 includes a body 13 for integrally wrapping legs, shoulders, a waist, arms, an upper body or a whole body and a connecting structure 14, and the body 13 is provided with the connecting structure 14.

It can be seen from the foregoing description that the carrier 1 adopts the part integral wrapping design, which can conveniently and rapidly wrap the legs, the waist, the arms, the upper body and the whole body to realize the integral cooling and heating therapy; and compared with a form of wearing the plurality of carriers 1 based on part regions in the prior art, the designed carrier 1 can be worn more conveniently. The integrally wrapped carrier 1 can be worn by the user himself/herself without professional guidance during wearing. Specifically, the connecting structure 14 can use a rapid disassembly and assembly design form such as zipper structure and magic sticker structure.

With reference to FIGS. 1 to 19, Embodiment I of the present application is as follows:
a cold and hot compress device including a carrier 1 and a main machine 2.

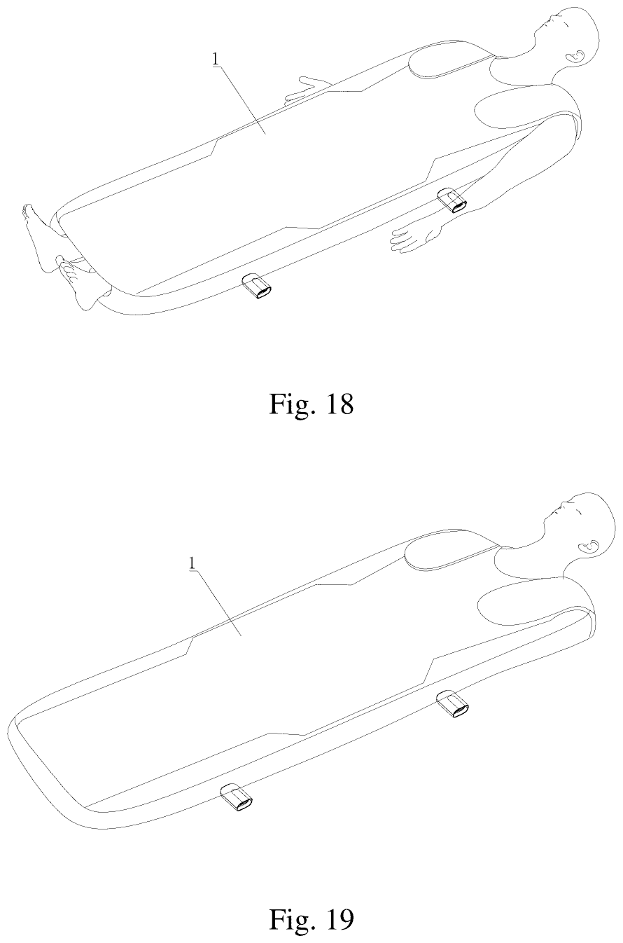

The carrier 1 includes an expansion layer 11 and a working layer 12, wherein the expansion layer 11 enables the carrier 1 to be closely adhered to the care site after being filled with a medium, and the working layer 12 is used for filling the medium after cooling and heating so as to make cold and hot compress on the adhered care site. The expansion layer 11 and the working layer 12 are both provided with a plurality of concave structures, so that after the expansion layer 11 and the working layer 12 can be in multi-point contact with the care site after being filled with a medium, and the expansion layer 11 and the working layer 12 are more coherent and can achieve a certain squeezing massage effect. In particular, as shown in FIGS. 14 to 19, the carrier 1 has components for wrapping the legs, the waist, the arms, the shoulders, and the neck, and the upper body as well as the whole body, and there are the leg binding carrier, the waist binding carrier, the arm binding carrier, the upper body binding carrier (for example, the carrier in a form of a wearable coat, for chest, back, shoulder and neck parts for nursing therapy) and the sleeping bag (capable of performing nursing therapy on the whole body) as the types of the formed carrier 1, which can be changed and used in a targeted manner. In particular, two styles of sleeping bags are shown in FIG. 18 and FIG. 19 respectively. The sleeping bag corresponding to FIG. 18 does not extend with parts for the arms and feet. The sleeping bag corresponding to FIG. 19 can wrap the parts including the arms and the feet, with the user only having the head exposed outside to realize more comprehensive cold and hot compress nursing.

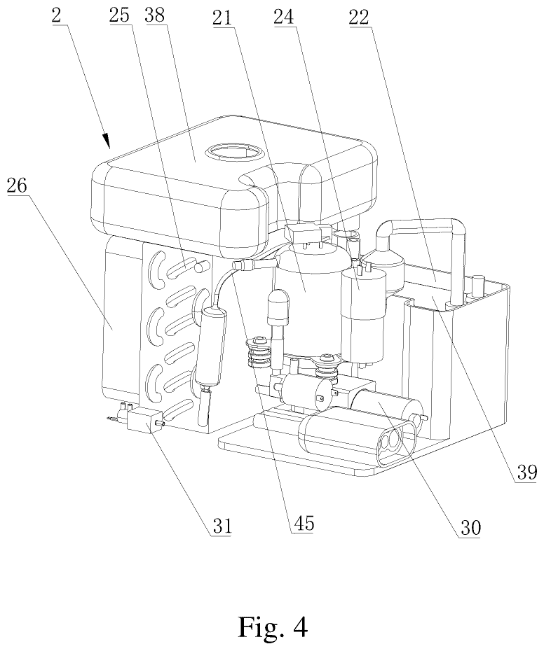

The main machine 2 is provided therein with a cooling and heating assembly, a circulation assembly, a heat dissipation assembly, a communication pipe 29, an air pump 30 and an air valve 31, wherein the cooling and heating assembly includes a compressor 21, a heat exchanger and a throttling element 45, and in the present embodiment, the heat exchanger includes a condenser 22 and an evaporator, and the throttling element 45 is an expansion valve. Alternatively, as shown in FIG. 4, the throttling element 45 may adopt a capillary tube, and the heat exchanger may adopt a plate type heat exchanger 43. The compressor 21, the condenser 22, the expansion valve and the evaporator are in communication in sequence; and the circulation assembly includes a storage tank, a pumping device 24 and a delivery pipe, wherein the pumping device 24 is connected to the storage tank and the carrier 1 via the delivery pipe, and the delivery pipe exchanges heat with the cooling and heating assembly. In particular, a pressure relief valve is also provided on the pumping device 24.

Specifically, the main machine 2 includes a housing and a mounting plate, wherein a bottom of the housing is provided with the mounting plate, and the cooling and heating assembly, the circulation assembly and the heat dissipation assembly are all locked onto the mounting plate. As shown in FIG. 1, the housing has an overall rectangular parallelepiped shape, a storage box is provided at one end of the housing in a length direction thereof, and the heat dissipation assembly and the cooling and heating assembly are provided at the other end of the housing in the length direction thereof. By designing the arrangement of the storage tank, the heat dissipation assembly and the cooling and heating assembly, the storage tank does not interfere with the heat dissipation of the cooling and heating assembly, and it is ensured that there is sufficient space for discharging the working heat of the main machine 2. The heat dissipation assembly and the cooling and heating assembly are arranged side by side in a width direction of the housing. The heat dissipation assembly includes a radiator 25 and a heat dissipation fan 26, wherein the radiator 25 is provided on a side face of the housing, and the heat dissipation fan 26 is provided between the radiator 25 and the cooling and heating assembly.

A power source 35 and a control circuit board 23 electrically connected to the power source 35, the cooling and heating assembly, the circulation assembly and the heat dissipation assembly are provided inside the main machine 2, a display screen 33 and a horn 34 are provided on an upper side of the main machine 2, and the display screen 33 and the horn 34 are also connected to the control circuit board 23 for observing operating conditions and alarming and prompting. A power source interface 36 and a switch 37 are provided on a back of the main machine 2.

The delivery pipe includes an internal circulation pipe 41, a first circulation pipe 27 and a second circulation pipe 28, the internal circulation pipe 41 is provided in the main machine 2, and the carrier 1, the pumping device 24 and the storage tank are all connected via the internal circulation pipe 41; the first circulation pipe 27 and the second circulation pipe 28 are connected to the inner circulation pipe 41 and the carrier 1; and the first circulation pipe 27 and the second circulation pipe 28 are double layered pipes including an inner layer and an outer layer, wherein the inner layer is hollow for a medium to pass through, and the outer layer is filled with a substance, such as air, which can be used to avoid temperature radiation of the inner layer. Using the design of a double-layered pipe can realize heat preservation of the media in the first circulation pipe 27 and the second circulation pipe 28.

The air pump 30 is connected to the air valve 31, the communication pipe 29 and the expansion layer 11 of the carrier 1, and the air pump 30 fills the expansion layer 11 of the carrier 1 with air so that the carrier 1 can abut against the care site.

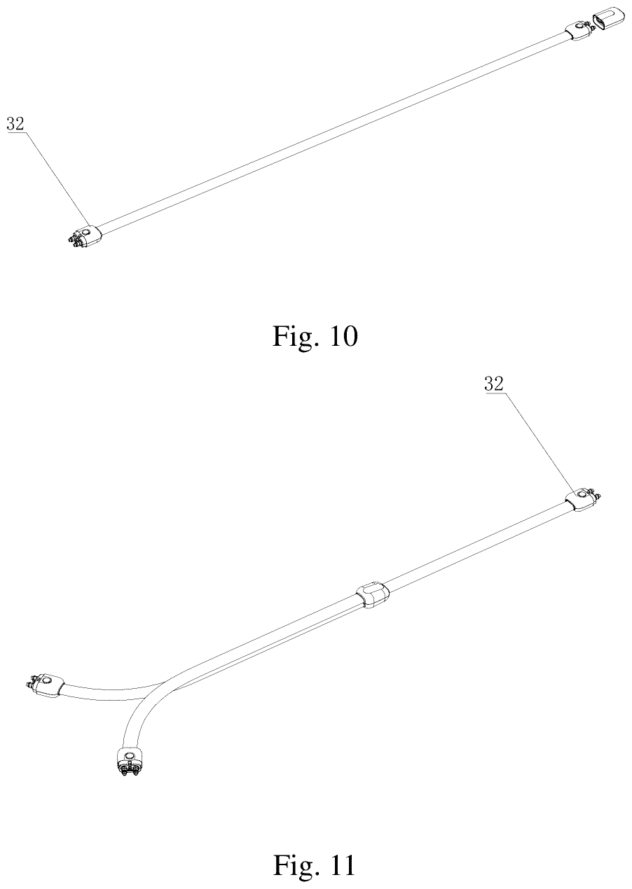

As shown in FIG. 10, the first circulation pipe 27, the communication pipe 29, and the second circulation pipe 28 are arranged side by side and integrally connected, so as to be conveniently assembled uniformly. The first circulation pipe 27, the second circulation pipe 28 and a middle portion of the communication pipe 29 are all provided with a quick connector 32, which can be extended or externally connected to a plurality of groups of carriers 1 according to needs. Specifically, as shown in FIG. 11, when the main machine is connected with a plurality of groups of carriers 1, a pipeline structure formed by combination of the first circulation pipe 27, the second circulation pipe 28 and the connection pipe 29 is shunted through a shunt joint.

### Embodiment II

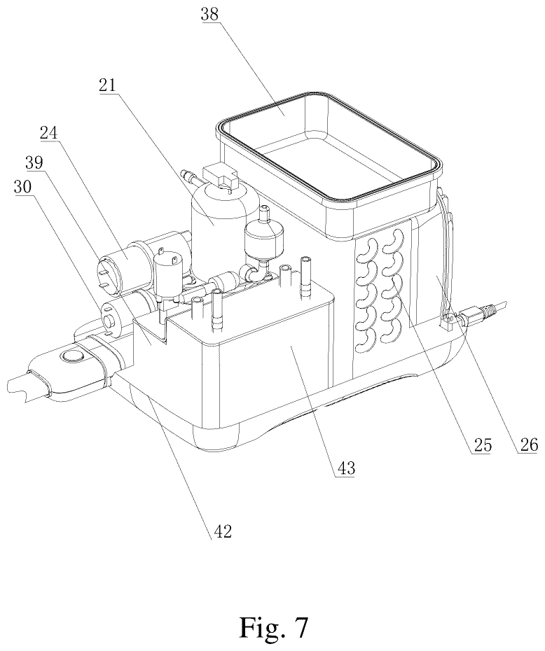

The difference between Embodiment II and Embodiment I is as follows: the cooling and heating assembly further includes an electric heating wire, which is used as an assembly for heating the medium in the delivery pipe, and the combination of the compressor 21, the condenser 22, the expansion valve and the evaporator is used for cooling the medium in the delivery pipe. In other embodiments, the cooling and heating assembly further includes a heating plate, which is used as the heating element 42. The heating plate can be provided on the delivery pipe side, the storage tank side or other parts that can conduct heat to the medium according to the mounting space inside the main machine. In FIG. 7, the heating element 42 is provided at the bottom of the storage tank.

### Embodiment III

The difference between Embodiment III and Embodiment I is as follows: a three-way valve 40 is provided in the main machine 2, and the air pump 30, the communication pipe 29, and the internal circulation pipe 41 are connected via the three-way valve 40. By the provision of the three-way valve 40, the air pump 30 can pump gas into the carrier 1 and thus evacuate the medium in the carrier 1.

### Embodiment IV

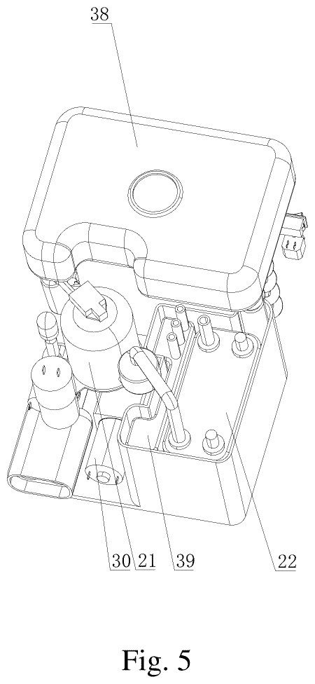

The difference between Embodiment IV and Embodiment I is as follows: as shown in FIGS. 4 and 5, the heat dissipation fan 26, the radiator 25, and the cooling and heating assembly are sequentially arranged along a length direction of the housing.

### Embodiment V

The difference between Embodiment V and Embodiment I is as follows: as shown in FIGS. 4 and 5, the storage tank includes a first tank 38 and a second tank 39, the first tank 38 is in communication with the second tank 39, and the second tank 39 is in communication with the pumping device 24 and the carrier 1. A mounting height of the first tank 38 is higher than a mounting height of the second tank 39. Specifically, a control valve is connected between the first tank 38 and the second tank 39, a water level detection device is provided in the second tank 39, and the water level detection device is electrically connected to the control valve. In addition, the principle of a communicating vessel can also be used to realize automatic supply in water shortage in the second tank 39 by using a water level difference between the first tank 38 and the second tank 39.

### Embodiment VI

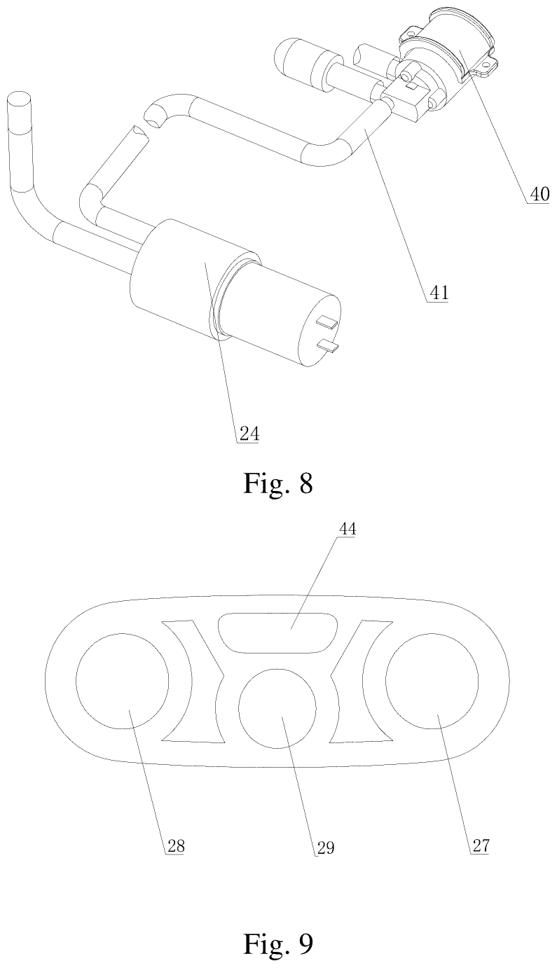

The difference between Embodiment VI and Embodiment III is as follows: as shown in FIG. 8, the pumping device 24 is a gas-liquid dual-purpose pump; a first end of the three-way valve 40 is connected with the storage tank; a second end of the three-way valve 40 is connected with the atmosphere; and a third end of the three-way valve 40 is connected with the pumping device 24. By combination of the design of the gas-liquid dual-purpose pump with the design of the three-way valve 40, a pumping function of the gas and the medium inside the carrier 1 can be realized, and the occupied mounting space of a plurality of pumping devices in the main machine 2 can be reduced.

### Embodiment VII

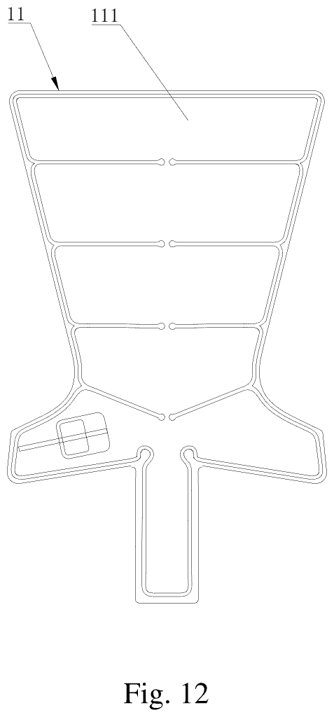

The difference between Embodiment VII and Embodiment I is as follows: as shown in FIG. 12, the expansion layer 11 includes a plurality of expansion units 111, each of which is connected with one air valve; and the plurality of air valves 31 are connected with the air pump 30. By the designs of the plurality of expansion units 111 and air valve 31, the human body part in a region covered by the carrier 1 can be regionally pressed by air pressure step by step, so that blood in the human body part can flow back rapidly, to improve a congestion state of the part after exercise, reduce fatigue after exercise, and accelerate repair of the part. In this embodiment, the carrier 1 can realize a multi-functional use, such as cold compress, hot compress, and inflatable binding to improve the fitting effect, and therapy on the congestion part by regional extrusion with the air pressure step by step.

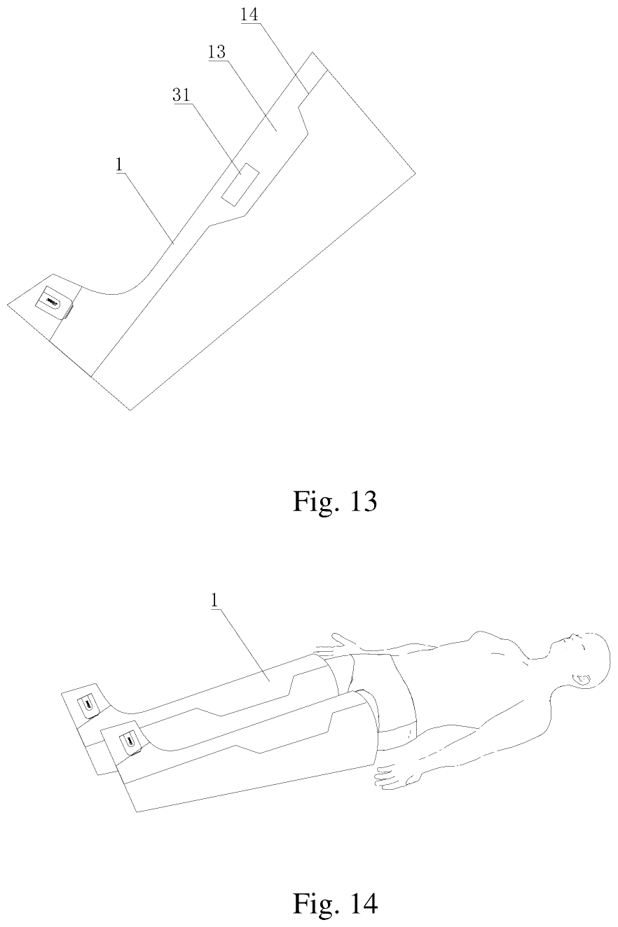

In other embodiments, as shown in FIG. 13, the air valves 31 are provided on the carrier. Through the arrangement of the air valves 31 on the carrier 1, mounting space occupation on the main machine 2 can be reduced, and a sufficient mounting space can be reserved inside the main machine 2 to configure other components. As shown in FIG. 9, accordingly, a control line 44 connected with the air valves 31 is provided in a pipeline structure formed by arranging side by side and integrally connecting the first circulation pipe 27, the communication pipe 29, and the second circulation pipe 28.

### Embodiment VIII

The difference between Embodiment VIII and Embodiment I is as follows: according to different functions of the matched carriers 1, the device can be of various styles. When the main machine 2 is matched with the carrier 1 without the congestion part therapy function, the main machine 2 under this style is provided with an air valve 31 inside, and the carrier 1 is not provided with the air valve 31. When the main machine 2 is only matched with the carrier 31 with the congestion part therapy function, the main machine 2 under this style is not provided with the air valve 31 inside, and the carrier 1 is provided with the air valve 31. When the main machine 2 can be matched with the carrier 1 without the congestion part therapy function and the carrier 1 with the congestion part therapy function at the same time, the main machine 2 under this style is provided with a main air valve inside, and the main air valve receives a signal from a control circuit board 23 for switching between opening and closing. In addition, the main air valve can also be in a normally open state, and another control assembly can perform opening and closing control on the main air valve. The carrier 1 with the congestion part therapy function is provided with a secondary air valve, and the carrier 1 without the congestion part therapy function is not provided with the air valve 31.

In summary, in the cold and hot compress device of the present application, through the arrangement of the circulation assembly, the medium is pumped by the storage tank via the pumping device, enters into the carrier to exchange heat with a care site after exchanging heat with the cooling and heating assembly, and finally returns to the storage tank; a temperature control of the medium in the delivery pipe is realized through cooling and heating by the cooling and heating assembly, so that a user can realize a cold and hot compress function after assembling the carrier; compared with traditional semiconductor cooling and heating, the cooling and heating assembly composed of the compressor, the evaporator, the expansion valve and the condenser has wider application scenes and a larger load, which can be applied to large-area and high-power cold and hot compress operation.

The foregoing description is merely an embodiment of the present application, and does not limit the scope of patent of the present application. Any equivalent transformation made by using the contents of the description and the drawings of the present application or direct or indirect use in the relevant technical field is likewise included in the scope of the patent protection of the present application.

1. A cold and hot compress device, comprising a carrier and a main machine, wherein a cooling and heating assembly and a circulation assembly are provided in the main machine, and the cooling and heating assembly comprises a compressor, a heat exchanger and a throttling element, wherein the compressor, the heat exchanger and the throttling element are in communication; and the circulation assembly comprises a storage tank, a pumping device and a delivery pipe, wherein the pumping device is connected to the storage tank and the carrier via the delivery pipe, and the delivery pipe exchanges heat with the cooling and heating assembly.

2. The cold and hot compress device according to claim 1, wherein a heat dissipation assembly is provided within the main machine, the heat dissipation assembly comprises a radiator and a heat dissipation fan, one side of the main machine is provided with the radiator, and one side of the radiator is provided with the heat dissipation fan.

3. The cold and hot compress device according to claim 2, wherein the storage tank comprises a first tank and a second tank, the first tank is in communication with the second tank, and the second tank is in communication with the pumping device and the carrier.

4. The cold and hot compress device according to claim 1, wherein the cooling and heating assembly further comprises a heating element configured for heating a medium in the delivery pipe.

5. The cold and hot compress device according to claim 1, wherein a channel of the delivery pipe comprises an internal circulation pipe, a first circulation pipe and a second circulation pipe, the internal circulation pipe is provided in the main machine, and the carrier, the pumping device and the storage tank are all connected via the internal circulation pipe; and the first circulation pipe and the second circulation pipe connect the inner circulation pipe and the carrier.

6. The cold and hot compress device according to claim 5, wherein the delivery pipe further comprises a communication pipe; an air pump is provided in the main machine, having one end connected with an air valve, and an expansion layer is provided on the carrier; and the air pump, the air valve and the expansion layer of the carrier are all connected via the communication pipe.

7. The cold and hot compress device according to claim 1, wherein a three-way valve is provided in the main machine; the pumping device is a gas-liquid dual-purpose pump; a first end of the three-way valve is connected with the storage tank; a second end of the three-way valve is connected with atmosphere; and a third end of the three-way valve is connected with the pumping device.

8. The cold and hot compress device according to claim 3, wherein a mounting height of the first tank is higher than a mounting height of the second tank.

9. The cold and hot compress device according to claim 6, wherein the expansion layer comprises a plurality of expansion units, each of which is connected with one air valve; and the plurality of air valves are connected with the air pump.

10. The cold and hot compress device according to claim 9, wherein the air valves are provided on the carrier.

11. The cold and hot compress device according to claim 1, wherein the carrier comprises a body for integrally wrapping legs, shoulders, a waist, arms, an upper body or a whole body and a connecting structure, and the body is provided with the connecting structure.
